(19) United States
(12) Patent Application Publication
(10) Pub. No.: US 2021/0133979 A1
(43) Pub. Date: May 6, 2021

(54) IMAGE PROCESSING APPARATUS, IMAGE PROCESSING METHOD, AND NON-TRANSITORY COMPUTER-READABLE STORAGE MEDIUM

(71) Applicant: CANON KABUSHIKI KAISHA, Tokyo (JP)

(72) Inventor: Naoto Takahashi, Kanagawa (JP)

(21) Appl. No.: 17/146,709

(22) Filed: Jan. 12, 2021

Related U.S. Application Data
(63) Continuation of application No. PCT/JP2019/024229, filed on Jun. 19, 2019.

(30) Foreign Application Priority Data
Aug. 10, 2018 (JP) 2018-151967

(51) Int. Cl. G06T 7/11; G16H 30/40; G16H 50/20; G06N 3/08

(52) U.S. Cl. CPC G06T 7/11; G16H 30/40; A61B 6/5211; G06N 3/08; G16H 50/20

(57) ABSTRACT

An image processing apparatus divides a radiological image obtained by performing radiography on a subject, into a plurality of anatomical regions, extracts at least one region from the plurality of anatomical regions, calculates a radiation dose index value for the radiography of the extracted region, based on a pixel value in the extracted region.

### CROSS-REFERENCE TO RELATED APPLICATIONS

This application is a Continuation of International Patent Application No. PCT/JP2019/024229. filed Jun. 19, 2019. which claims the benefit of Japanese Patent Application No. 2018-151967, filed Aug. 10, 2018, both of which are hereby incorporated by reference herein in their entirety.

### BACKGROUND OF THE INVENTION

### Field of the Invention

The present invention relates to an image processing technique for outputting a radiation dose index value based on an image obtained through radiography.

### Background Art

Recent years have seen growing use of digital images in the medical field. Using a digital radiographic apparatus (radiation shooting apparatus) that uses a sensor for indirectly or directly converting radiation (X-rays) into electrical signals in order to generate a digital image has been a main trend.

This radiographic apparatus has a very wide dynamic range for a radiation dose, and also has an advantage that, even when there is a deficiency or excess in the radiation dose, stable-density output is obtained through automatic density correction that is performed through image processing, compared with conventional analog radiography. On the other hand, even when a radiographing operator performs shooting with an inappropriate radiation dose, he or she is unlikely to notice that, and, in particular, when the radiation dose is excessive, there is the issue that a patient's exposure amount increases.

In view of this, in order to solve this issue, a value that is a standard of a radiation dose for shooting a digital radiological image (hereinafter, referred to as a “radiation dose index value”) is usually displayed along with a shot image. In addition, various methods for calculating a radiation dose index value have been proposed. Recently, the international standard IEC62494-1 was issued by IEC (International Electrotechnical Commission), and EI (Exposure Index) was defined as a standardized radiation dose index value. In addition, in this international standard, EIT (Target Exposure Index) is determined as the value of a radiation dose (hereinafter, referred to as a “radiation dose target value”) that is to be a target, and a method for conducting radiation dose management using DI (Deviation Index) indicating the difference between the radiation dose index value EI and the radiation dose target value EIT is also provided. Manufacturers provide radiation dose management functions conforming to this international standard. Manufacturers have proposed various calculation methods such as those in Patent Documents 1 and 2.

### CITATION LIST

### Patent Literature

PTL1: Japanese Patent Laid-Open No. 2014-158580

PTL1: Japanese Patent Laid-Open No. 2015-213546

A method for calculating a radiation dose index value EI has been a black box in most cases. Therefore, numerical implication of the radiation dose index value EI is not clear to the operator, and has been inconvenient when used as a reference value of radiation dose management.

This disclosure provides a technique for performing more appropriate radiation dose management using a reference intended by the operator.

### SUMMARY OF THE INVENTION

According to one aspect of the present invention, there is provided an image processing apparatus which includes: a division unit configured to divide a radiological image obtained by performing radiography on a subject, into a plurality of anatomical regions; an extraction unit configured to extract at least one region from the plurality of anatomical regions and a calculation unit configured to calculate a radiation dose index value for the radiography of the region extracted by the extraction unit, based on a pixel value in the extracted region.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 shows a configuration example of a radiographic apparatus according to a first embodiment.

FIG. 2 is a flowchart showing the processing procedure of a radiographic apparatus 100 according to the first embodiment.

FIG. 3 is a flowchart showing the processing procedure for changing a region for calculating a radiation dose index value according to the first embodiment.

FIG. 4 shows a configuration example of the radiographic apparatus according to the first embodiment.

FIG. 5 is a flowchart showing the processing procedure for changing a division configuration for a region for calculating a radiation dose index value according to a second embodiment.

FIG. 6 shows a configuration example of a radiographic apparatus according to a third embodiment.

FIG. 7 is a flowchart showing the processing procedure for automatically updating a radiation dose target value EIT according to the third embodiment.

FIG. 8A is a diagram showing an example of a segmentation map according to the first embodiment.

FIG. 8B is a diagram showing an example of a changed correct-answer segmentation map according to the second embodiment.

FIG. 9 is a diagram showing an example of a correspondence table of the correspondence between a plurality of shooting sites and label numbers.

### DESCRIPTION OF THE EMBODIMENTS

The present invention will be described in detail below with reference to the accompanying drawings based on exemplary embodiments. Note that configurations described in the following embodiments are merely exemplary, and the present invention is not limited to the illustrated configurations.

### First Embodiment

### Configuration of Radiographic Apparatus

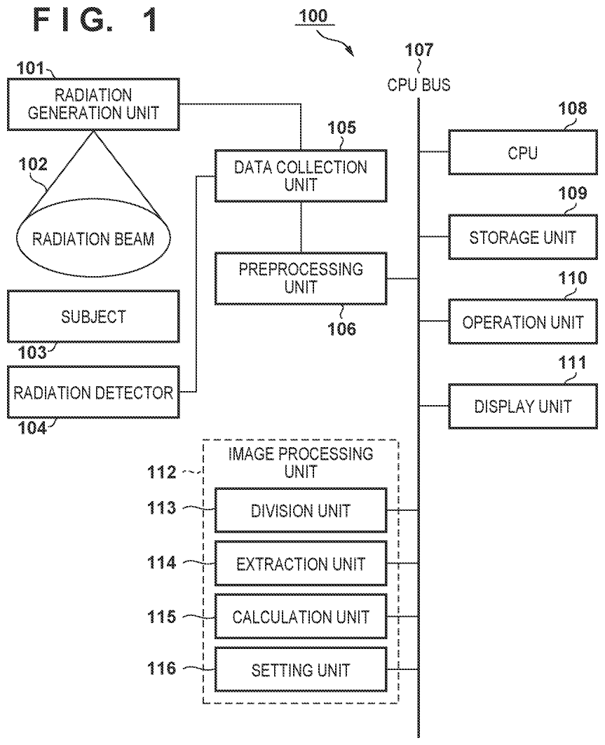

FIG. 1 shows a configuration example of a radiographic apparatus 100 according to a first embodiment. The radiographic apparatus 100 is a radiographic apparatus that has an image processing function for outputting a radiation dose index value based on an image obtained through radiography. Accordingly, the radiographic apparatus 100 also functions as an image processing apparatus. The radiographic apparatus 100 includes a radiation generation unit 101, a radiation detector 104, a data collection unit 105, a preprocessing unit 106, a CPU (Central Processing Unit) 108, a storage unit 109, an operation unit 110, a display unit 111, and an image processing unit 112, and these units are connected such that data can be mutually transmitted/received via a CPU bus 107. In addition, the image processing unit 112 includes a division unit 113, an extraction unit 114, a calculation unit 115, and a setting unit 116. The storage unit 109 stores various types of data and control programs required for processing to be performed by the CPU 108, and functions as a working memory of the CPU 108. The CPU 108 performs operation control and the like of the entire apparatus in accordance with an operation from the operation unit 110, using the storage unit 109. Accordingly, the radiographic apparatus 100 operates in a manner to be described later.

First, when a shooting instruction is input by the operator via the operation unit 110, this shooting instruction is transmitted to the radiation generation unit 101 and the radiation detector 104 via the data collection unit 105 by the CPU 108. Subsequently, the CPU 108 controls the radiation generation unit 101 and the radiation detector 104 to execute radiography. In the radiography, first, the radiation generation unit 101 irradiates a subject 103 with a radiation beam 102. The radiation beam 102 emitted from the radiation generation unit 101 passes through the subject 103, and reaches the radiation detector 104. The radiation detector 104 then outputs signals that are based on the intensity (radiation intensity) of the radiation beam 102 that reached the radiation detector 104. Note that, according to this embodiment, the subject 103 is a human body. Thus, the signals that are output from the radiation detector 104 are data obtained by shooting a human body.

The data collection unit 105 converts the signals output from the radiation detector 104 into predetermined digital signals, and supplies the digital signals as image data to the preprocessing unit 106. The preprocessing unit 106 performs preprocessing such as offset correction and gain correction on the image data supplied from the data collection unit 105. The image data subjected to preprocessing by the preprocessing unit 106 is sequentially transferred to the storage unit 109 and the image processing unit 112 via the CPU bus 107 under control by the CPU 108.

The image processing unit 112 performs processing for calculating a radiation dose index value based on the image data (hereinafter, “radiological image”) obtained from the preprocessing unit 106. The radiation dose index value is a value that is a standard of a radiation dose for shooting as described above. The division unit 113 divides the radiological image (radiological image obtained by performing radiography on the subject 103) that has been input thereto, into a plurality of anatomical regions. According to this embodiment, the division unit 113 creates a segmentation map (multivalued image) to be described later. The extraction unit 114 extracts at least one region from among the plurality of anatomical regions divided by the division unit 113, as a region for calculating a radiation dose index value, based on an operator's operation on the operation unit 110. The calculation unit 115 calculates a radiation dose index value for performing radiography on the region extracted by the extraction unit 114, based on pixel values in the extracted region. The setting unit 116 sets and manages information regarding the correspondence between label number and training data or shooting site, which will be described later.

The radiation dose index value calculated by the image processing unit 112 is displayed on the display unit 111 along with the radiological image obtained from the preprocessing unit 106 under control by the CPU 108. After the operator confirms the radiation dose index value and radiological image displayed on the display unit 111, a series of shooting operations end. The radiation dose index value and radiological image displayed on the display unit 111 may also be output to a printer or the like (not illustrated).

### Operations of Radiographic Apparatus

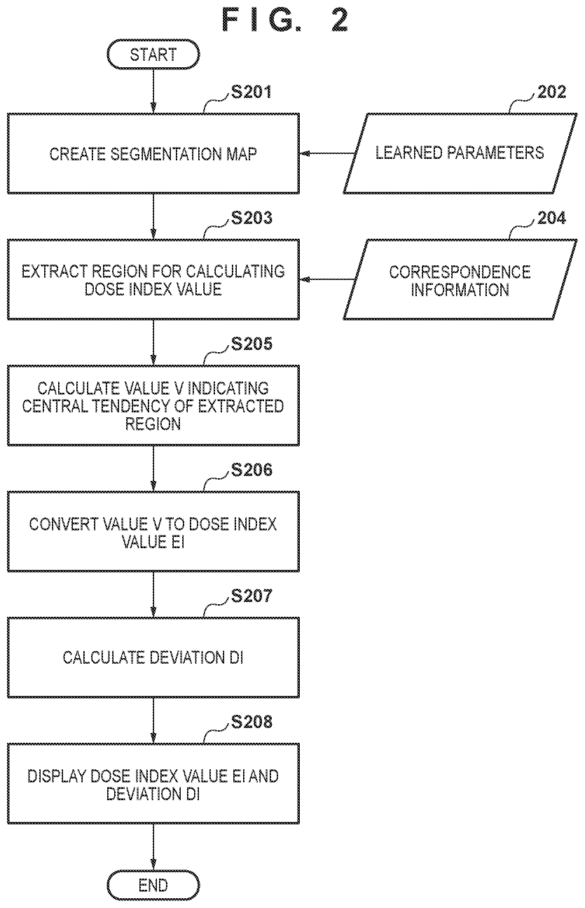

Next, operations of the radiographic apparatus 100 according to this embodiment will be described in detail with reference to the flowchart in FIG. 2. FIG. 2 is a flowchart showing the processing procedure of the radiographic apparatus 100 according to this embodiment. The flowchart shown in FIG. 2 can be realized as a result of the CPU 108 executing a control program stored in the storage unit 109, and executing computation and processing of information as well as control of items of hardware.

As described above, the radiological image obtained by the preprocessing unit 106 is transferred to the image processing unit 112 via the CPU bus 107. The transferred radiological image is input to the division unit 113. The division unit 113 creates a segmentation map (multivalued image) from the input radiological image (step S201). Specifically, the division unit 113 adds, to each pixel of the radiological image, a label indicating an anatomical region to which that pixel belongs. Such division of an image into any anatomical regions is called semantic segmentation (semantic region division). Note that, according to this embodiment, the label is a label number distinguishable by the pixel value. Specifically, the division unit 113 divides a radiological image into a plurality of anatomical regions by adding different label numbers to the plurality of anatomical regions.

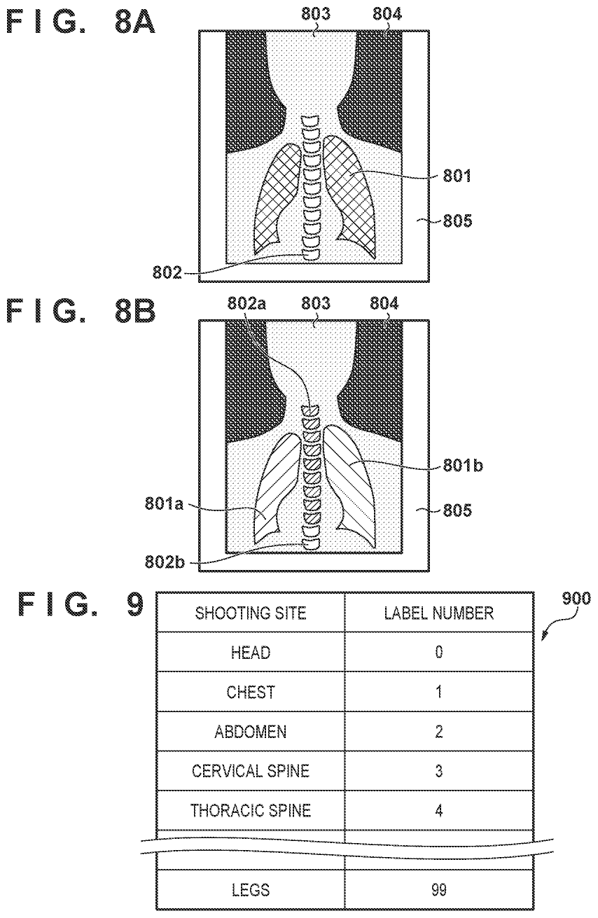

FIG. 8A shows an example of the segmentation map created in step S201. FIG. 8A is a diagram showing an example of a segmentation map when an input radiological image is an image that shows a chest. As shown in FIG. 8A, the division unit 113 provides the same value (e.g., a pixel value of 0) to the pixels of a region that belongs to a lung field 801, and provides the same value (e.g., a pixel value of 1) different from the above value of the lung field to the pixels of a region that belongs to a spine 802. In addition, the division unit 113 provides the same value (e.g., a pixel value of 2) different from the above values of the lung field and spine to the pixels that belong to a subject structure 803 that does not belong to any of the lung field 801 and the spine 802.

Note that division shown in FIG. 8A is exemplary, and the granularity according to which a radiological image is divided into anatomical regions is not limited in particular. For example, the division unit 113 may determine the granularity of division as appropriate according to a desired region for calculating a radiation dose index value instructed by the operator via the operation unit 110. In addition, the division unit 113 may also create a segmentation map by adding label numbers (pixel values) to regions other than the subject structure in a similar manner. For example, in FIG. 8A, it is also possible to create a segmentation map in which different label numbers are added to a region 804 in which radiation directly reaches the radiation detector 104 and a region 805 in which radiation is shielded by a collimator (illustrated).

According to this embodiment, the division unit 113 creates a segmentation map in step S201 using a known method. Here, a CNN (Convolutional Neural Network) is used as an example of the known method. A CNN is a neural network constituted by a convolution layer, pooling layer, full-connected layer, and the like, and is realized by appropriately combining such layers according to a problem to be solved. In addition, a CNN represents a type of a machine learning algorism, and requires prior training. Specifically, a filter coefficient used for a convolutional layer and parameters (variables) such as a weight and bias value of each layer need to be adjusted (optimized) through so-called supervised learning that uses a large number of pieces of training data.

Here, supervised learning includes preparation of a large number of samples of combination of an image that is input to the CNN (input image) and an output result (correct answer) expected when that input image is provided, and repeated adjustment of parameters so as to output an expected result. The error backpropagation method (back propagation) is commonly used for this adjustment. Specifically, parameters of the CNN are repeatedly adjusted in a direction in which the difference between the correct answer and actual output result (error defined by a loss function) decreases.

Note that, according to this embodiment, an image that is input to the CNN is a radiological image obtained by the preprocessing unit 106, and an expected output result is a correct-answer segmentation map (e.g., FIG. 8A). Such a correct-answer segmentation map can be created by the operator in accordance with the granularity of a desired anatomical region in advance. CNN parameters (learned parameters 202) are generated in advance through machine learning using a plurality of samples of combination of an input image and an expected output result. The learned parameters 202 are stored in the storage unit 109 in advance, and, when the division unit 113 creates a segmentation map in step S201, the learned parameters 202 are called, and semantic segmentation is performed using the CNN. Accordingly, the division unit 113 divides the radiological image into a plurality of anatomical regions using parameters generated through machine learning in advance. In addition, the operator can determine, as training data, predetermined image data and a correct-answer segmentation map corresponding to the predetermined image data (division/allocation data). The training data is managed by the setting unit 116.

Note that only one learned parameter 202 may also be generated using data of all of the sites, but a learned parameter 202 may also be generated for each site (e.g., head, chest, abdomen, four extremities). Specifically, a configuration may also be adopted in which learning is separately performed using a plurality of samples of combinations of an input image and an expected output result for each site, and thereby a plurality of sets of learned parameters 202 are generated for each site. A configuration may also be adopted in which, when a plurality of sets of learned parameters 202 are generated, learned parameters of each set are stored in the storage unit 109 in advance in association with site information, and the division unit 113 calls learned parameters 202 corresponding to a shooting site from the storage unit 109 according to a site of an input image, and perform semantic segmentation using the CNN.

Note that the network structure of the CNN is not particularly limited, and a generally known structure may be used. Specifically, FCN (Fully Convolutional Networks), SegNet, U-net, or the like can be used for machine learning. In addition, according to this embodiment, an image that is input to the CNN is a radiological image obtained by the preprocessing unit 106, but a radiological image obtained by reducing such a radiological image may also be used as an image that is input to the CNN. Semantic segmentation that uses a CNN requires a large calculation amount and a long calculation time, and thus use of reduced image data can lead to a reduction in the calculation time.

Next, the extraction unit 114 extracts a region for calculating a radiation dose index value (step S203). Specifically, the extraction unit 114 extracts, as a region for calculating a radiation dose index value, a region specified by the operator via the operation unit 110. According to this embodiment. in order to perform the extraction processing, correspondence information 204 that is information regarding the correspondence between a shooting site and a label number is used as region information. Specifically, the extraction unit 114 extracts at least one region out of a plurality of anatomical regions using the correspondence information 204 that is information regarding the correspondence between a plurality of sites and label numbers respectively corresponding to the plurality of sites, in accordance with an operator's instruction, the correspondence information 204 having been set in advance. FIG. 9 shows, as an example of the correspondence information 204, an example of a table of the correspondence between a plurality of shooting sites and label numbers respectively corresponding to the plurality of sites. A correspondence table 900 shown in FIG. 9 is created by the operator or the like in advance, and is stored in the storage unit 109. The extraction unit 114 references the correspondence table 900, and obtains a label number corresponding to the site of a region designated (instructed) by the operator via the operation unit 110. Note that the extraction unit 114 may also directly obtain a label number specified by the operator via the operation unit 110. Subsequently, the extraction unit 114 generates a mask image (Mask) in which the value of a pixel corresponding to the obtained label number is 1, based on Expression 1.

$$Mask ( x , y ) = \{ \begin{matrix}1 , & Map ( x , y ) = L \\ 0 , & Map ( x , y ) \neq L\end{matrix} \qquad ( 1 )$$

Note that Map indicates a segmentation map, and (x,y) indicates coordinates in an image. Also, L indicates an obtained label number.

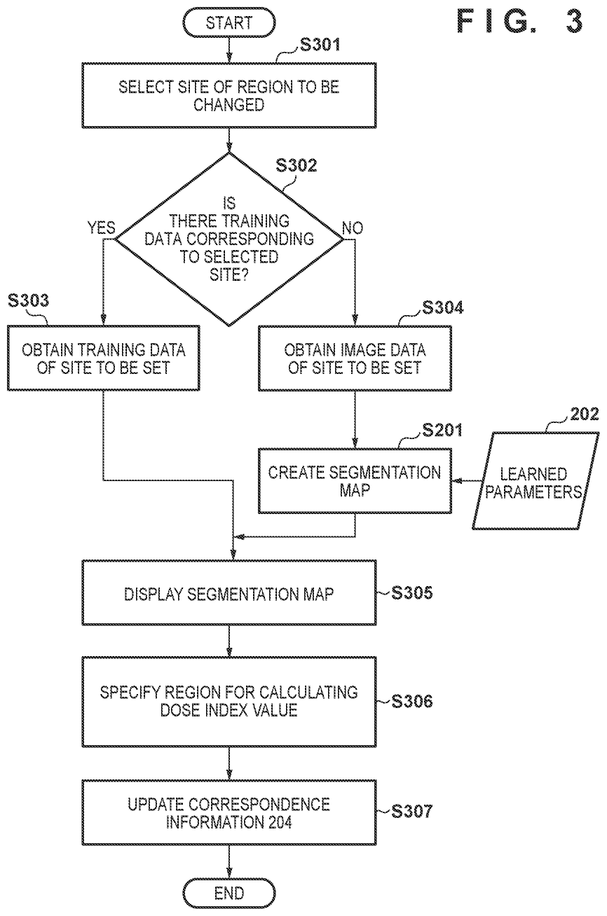

Note that the number of regions specified by the operator is not limited to one, and a plurality of regions may also be specified. When the operator specifies a plurality of regions, the extraction unit 114 may generate a mask image in which the value of a pixel corresponding to one of a plurality of label numbers corresponding to a plurality of region is 1, and the value other than that is 0. Note that a method for the operator to set a region will be described in detail later with reference to the flowchart in the FIG. 3.

Next, the calculation unit 115 calculates a value V indicating the central tendency of the extracted region, as a representative value in the region extracted in step S203 (i.e., a region in the mask image in which the pixel value is 1) (step S205). According to this embodiment, the value V is calculated as in Expression 2.

$$V = \frac{{\sum }_{x} {\sum }_{y} Org ( x , y ) \cdot Mask ( x , y )}{{\sum }_{x} {\sum }_{y} Mask ( x , y )} \qquad ( 2 )$$

Note that Org indicates an input image (according to this embodiment, a radiological image obtained by the preprocessing unit 106), Mask indicates a mask image, (x,y) indicates coordinates in the image, and Org (x,y) indicates a pixel value at coordinates (x,y) in the input image.

Next, the calculation unit 115 converts the obtained value V into a radiation dose index value EI (step S206). Specifically, the calculation unit 115 converts the value V into a radiation dose index value EI in accordance with the definition of international standard TEC62494-1 as in Expression 3.

EI=c₀·g(V),c₀=100μGy⁻¹ (3)

Note that a function g is a function for converting the value V into air kerma, and is determined in advance in accordance with the relationship between the air kerma and the value V under a stipulated condition. Note that the function g differs according to the property of the radiation detector 104. Therefore, the operator stores a plurality of functions g in the storage unit 109 in advance in correspondence with available radiation detectors 104, such that the calculation unit 115 can perform conversion using a function g corresponding to a radiation detector 104 that is actually used.

In addition, the calculation unit 115 calculates the difference amount (deviation) DI between a radiation dose target value EIT and the radiation dose index value EI, using Expression 4 (step S207).

$$DI = 10 \cdot {log}_{10} ( \frac{E I}{{EI}_{T}} ) \qquad ( 4 )$$

Note that the radiation dose target value EIT is set in advance for each site in accordance with a radiation dose management reference determined by the operator. The deviation DI is a numerical value indicating the deviation between the radiation dose target value EIT and the radiation dose index value EI, and thus, if the radiation dose target value EIT and the radiation dose index value EI are the same, the deviation DI is 0. In addition, the larger the radiation dose index value EI is, in other words the larger the extent to which the radiation dose of a shot image is larger than the radiation dose target value EIT, the larger the deviation D becomes. For example, when the radiation dose of a shot image is twice as large as the radiation dose target value EIT, the deviation DI is about 3. In addition, the smaller the extent to which the radiation dose of a shot image is smaller than the radiation dose target value EIT, the smaller the deviation DI becomes, and, for example, when the radiation dose of a shot image is half the radiation dose target value, the deviation DI is about −3. Accordingly, the operator can instantaneously understand whether the radiation dose of a shot image is deficient or excessive relative to the radiation dose target value EIT that is a reference.

Next, the CPU 108 displays the obtained the radiation dose index value EI and deviation DI, on the display unit 111 (step S208). Here, the display method is not particularly limited, and, for example, the CPU 108 can perform the display on the display unit Ill along with the radiological image obtained by the preprocessing unit 106, and the CPU 108 may also perform control so as to display the obtained radiation dose index value EI and deviation DI as an overlay on a portion of the display area on which the display unit 111 can perform display.

Note that, according to this embodiment, only one radiation dose index value EI and only one deviation DI are calculated based on a region specified by the operator, but, for example, a configuration may also be adopted in which a plurality of radiation dose index values EI and deviations DI are obtained. Specifically, a configuration may also be adopted in which, when a plurality of regions are extracted as a result of the operator specifying these regions, the radiographic apparatus 100 calculates a value D for each of the regions, calculates the radiation dose index value EI and the deviation DI for the value D, and display such data on the display unit 111.

An operation of calculating a radiation dose index value based on a radiological image obtained through shooting has been described above. Next, operations of the image processing unit 112 when the operator changes a region for calculating a radiation dose index value will be described with reference to the flowchart in FIG. 3. FIG. 3 is a flowchart showing the processing procedure for changing a region for calculating a radiation dose index value. The flowchart shown in FIG. 3 can be realized as a result of the CPU 108 executing a control program stored in the storage unit 109, and executing computation and processing of information as well as control of each item of hardware. Note that, in the flowchart in FIG. 3, the same reference signs are assigned to the same steps as the steps shown in FIG. 2, and a description thereof is omitted.

First, the operator selects the site of a region (site to which this region belongs) to be changed via the operation unit 110 (step S301). Next, the setting unit 116 determines whether or not there is training data corresponding to the selected site (step S302). Here, as described above, training data refers to a correct-answer segmentation map determined by the operator (division/allocation data). If there is training data corresponding to the selected site (Yes in step S302), the setting unit 116 obtains the training data from the storage unit 109 (step S303). If there is no training data corresponding to the selected site (No in step S302), the setting unit 116 obtains, from the storage unit 109. image data (radiological image) of the selected site obtained through past shooting (step S304), and the division unit 113 creates a segmentation map from the obtained image data (step S201). A method for creating a segmentation map is the same as that in the description on step S201 in FIG. 2.

Next, the CPU 108 displays a segmentation map such as that shown in FIG. 8A, on the display unit 111 (step S305). The operator specifies a region to be changed, which is any region for calculating a radiation dose index value, using a mouse or the like (not illustrated) (step S306). Next, the setting unit 116 obtains a label number corresponding to the specified region, updates the correspondence information 204 (e.g., the correspondence table 900 of the correspondence between shooting sites and label numbers shown in FIG. 9), and stores (sets) the data in the storage unit 109 (step S307).

As described above, according to the first embodiment, it is possible to freely change a region for calculating a radiation dose index value from among regions obtained by dividing a radiological image, and to perform appropriate radiation dose management using a reference intended by the operator.

### Second Embodiment

### Configuration of Radiographic Apparatus

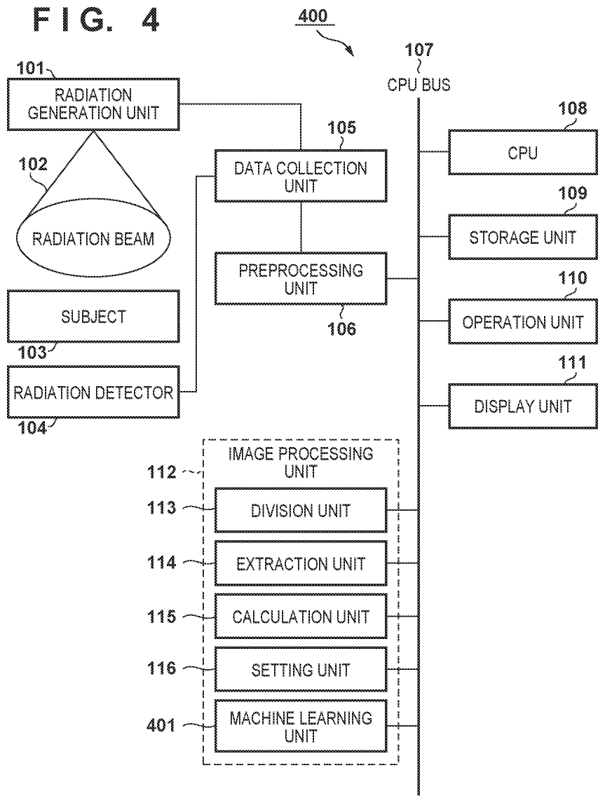

FIG. 4 shows a configuration example of a radiographic apparatus 400 according to a second embodiment. The radiographic apparatus 400 has a configuration of the radiographic apparatus 100 shown in FIG. 1 that includes a machine learning unit 401. The machine learning unit 401 has a function of performing leaning for changing a division configuration for a region for calculating a radiation dose index value. Specifically, the machine learning unit 401 performs machine learning (CNN relearning) based on training data (predetermined image data and a correct-answer segmentation map (division/allocation data) corresponding to the predetermined image data).

### Operations of Radiographic Apparatus

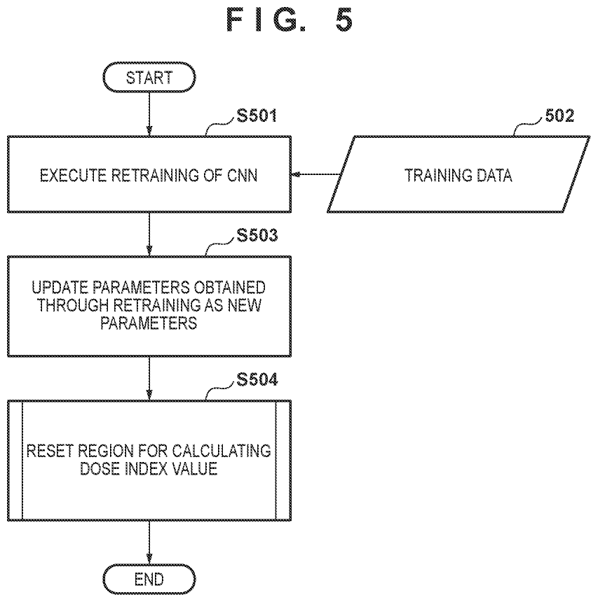

Processing for changing the division configuration for a region for calculating a radiation dose index value, which is an operation different from that in the first embodiment, will be described below with reference to FIG. 5. FIG. 5 is a flowchart showing the processing procedure for changing the division configuration for a region for calculating a radiation dose index value. The flowchart shown in FIG. 5 can be realized as a result of the CPU 108 executing a control program stored in the storage unit 109, and executing computation and processing of information as well as control of each item of hardware.

First, the machine learning unit 401 retrains the CNN based on training data 502 (step S501). This retraining is training that uses the training data 502 prepared in advance. Note that a specific training method is performed by repeatedly adjusting parameters of the CNN in a direction in which the difference between the correct answer and actual output result (error defined by a loss function) decreases, using the error backpropagation method (back propagation) similarly to that described in the first embodiment.

Here, the setting unit 116 can set training data for the machine learning unit 401 to perform retraining, as will be described below. Specifically, the setting unit 116 can change the correct-answer segmentation map that is training data, and set the correct-answer segmentation map in the machine learning unit 401. FIG. 8B shows an example of the changed correct-answer segmentation map. For example, according to the first embodiment, the division unit 113 adds the same label to the lung field 801 as one region as shown in FIG. 8A. On the other hand, according to this embodiment, as shown in FIG. 8B, the setting unit 116 prepares, as the training data 502, a correct-answer segmentation map in which different labels are added to a right lung field 801a and a left lung field 801b, in consideration of a case where the lung field is divided into different regions, namely a right lung field and a left lung field. In addition, according to the first embodiment, the division unit 113 adds the same label to the spine 802 as one region, as shown in FIG. 8A. On the other hand, according to this embodiment, the setting unit 116 prepares a correct-answer segmentation map in which different labels are added to a dorsal vertebra 802a and a lumbar vertebra 802b as shown in FIG. 8B, in consideration of a case where the spine is divided into a dorsal vertebra and a lumbar vertebra.

Next, the machine learning unit 401 updates (stores) parameters obtained through retraining as new parameters of the CNN, in the storage unit 109 (step S503). Subsequently, the image processing unit 112 resets a region for calculating a radiation dose index value (step S504). Here, the resetting method is the same as in the operation of the flowchart in FIG. 3, and thus a description thereof is omitted. Note that, in FIG. 3, as a result of using new training data in the above processing, a new segmentation map is created, and, in the process in step S307, the correspondence information 204 (e.g., the correspondence table 900 of the correspondence between shooting sites and label numbers shown in FIG. 9) is updated. Specifically, the setting unit 116 updates and sets the correspondence information 204 according to a plurality of anatomical regions obtained through division using the updated parameters. Accordingly, for example, in shooting for the next time onward (FIG. 2), as a result of replacing the learned parameters 202 (FIG. 2) with the parameters updated in step S503, the division unit 113 can divide a radiological image into a plurality of anatomical regions using the updated parameters. Furthermore, it is possible to calculate a radiation dose index value for a newly defined region by using the correspondence information 204 updated according to this embodiment in place of the correspondence information 204 that is used in step S203.

As described above, according to the second embodiment, there are effects that it is possible to change a division configuration for a region for calculating a radiation dose index value, and the operator can freely change the region for calculating a radiation dose index value.

### Third Embodiment

### Configuration of Radiographic Apparatus

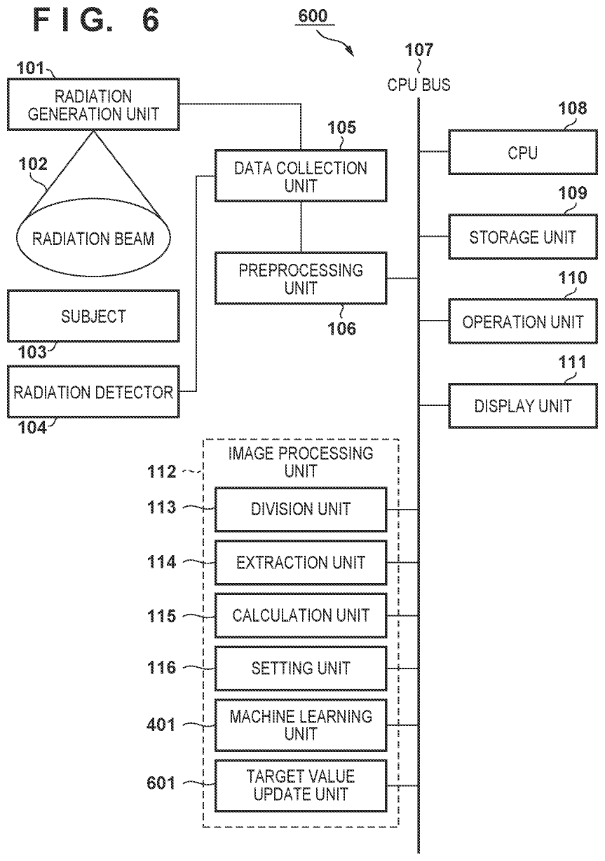

FIG. 6 shows a configuration example of a radiographic apparatus 600 according to a third embodiment. The radiographic apparatus 600 has a configuration of the radiographic apparatus 400 shown in FIG. 4 that includes a target value update unit 601. The target value update unit 601 has a function of automatically setting the radiation dose target value EIT.

The radiation dose target value EIT is a value that is a reference for the radiation dose index value EI, and, when a region for calculating the radiation dose index value EI changes, the radiation dose target value EIT also needs to be changed. This change is manually set by the operator in accordance with the radiation dose management reference, but, setting a region for calculating the radiation dose index value EI from scratch every time a region for calculating the radiation dose index value EI is changed is very troublesome. In view of this, the target value update unit 601 has a function for automatically updating the radiation dose target value EIT based on the difference of the region before and after change, using a value that is substantially equal to the radiation dose target value EIT before being changed.

### Operation of Radiographic Apparatus

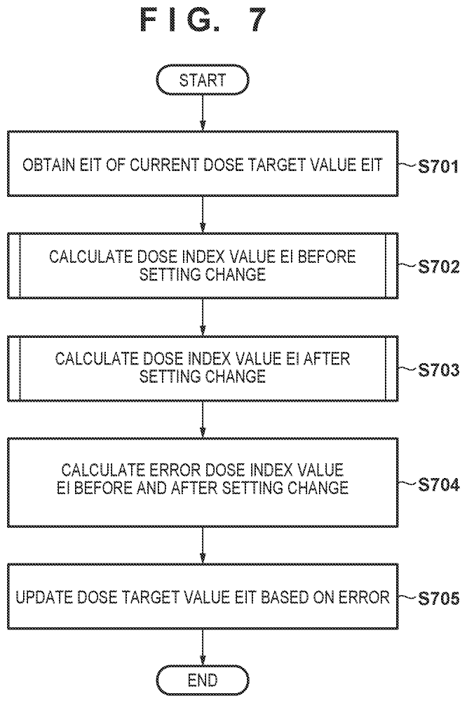

A method for automatically updating the radiation dose target value EIT will be described below with reference to FIG. 7. FIG. 7 is a flowchart showing the processing procedure for automatically updating the radiation dose target value EIT. The flowchart shown in FIG. 7 can be realized as a result of the CPU 108 executing a control program stored in the storage unit 109, and executing computation and processing of information as well as control of each item of hardware. Note that this flowchart is performed at a timing when a region for calculating a radiation dose target value is changed. Specifically, this flowchart is executed after when the region for calculating a radiation dose index value is changed (operation that is based on the flowchart in FIG. 3), or the division configuration for a region for calculating a radiation dose index value is changed (operation that is based on the flowchart in FIG. 5).

First, the target value update unit 601 obtains EIT that is currently set for a region for calculating a radiation dose target value (step S701). Next, the calculation unit 115 loads, from the storage unit 109, a plurality of radiological images obtained through past shooting, and calculates the radiation dose index value EI for each of the radiological images (step S702). Here, a method for calculating the radiation dose index value EI is the same as that described with reference to the flowchart in FIG. 2. Note that the parameter 202 learned when calculating the radiation dose index value EI and the correspondence information 204 that are used are the parameter 202 learned when calculating the radiation dose index value EI and the correspondence information 204, which have not been subjected to a setting change (change in the region for calculating a radiation dose index value (FIG. 3) or change in the division configuration for a region for calculating a radiation dose index value (FIG. 5)). Next, the calculation unit 115 calculates the radiation dose index value EI subjected to a setting change similarly to step S702 (step S703). Difference from step S702 is that the learned parameter 202 and the correspondence information 204 after a setting change are used.

Next, an error Err of EI before and after a setting change is calculated based on Expression 5 (step S704).

$$Err = \frac{1}{N} \cdot {\sum }_{k = 1}^{N} ( {EI}_{1} ( k ) - {EI}_{2} ( k ) ) \qquad ( 5 )$$

Note that k indicates an image number, and N indicates the total number of images for which EI was calculated. In addition, EI1 (k) and EI2 (k) are EIs calculated based on the image of the image number k, EI1 indicating E before a setting change, and EI2 indicating EI after a setting change.

Next, the radiation dose target value EIT is updated using the obtained error Err (step S705).

EI_{T}2=EI_{T}1+Err (6)

Note that EIT1 indicates a radiation dose target value before update, and EIT2 indicates a radiation dose target value after update.

In this manner, when the learned parameter 202 is updated, or when the correspondence information 204 is updated, the target value update unit 601 updates a radiation dose target value that is a target value of a radiation dose index value, using radiation dose index values calculated by the calculation unit 115 before and after the update. As described above, according to the third embodiment, there is an effect that the trouble of the operator can be reduced by automatically updating the value of EIT during a setting change.

Although several embodiments have been described above, it is needless to say that the present invention is not limited to these embodiments, and various modifications and changes can be made within the scope of the gist.

According to the present invention, it is possible to perform more appropriate radiation dose management using a reference intended by the operator.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a division unit configured to divide a radiological image obtained by performing radiography on a subject, into a plurality of anatomical regions;
an extraction unit configured to extract at least one region from the plurality of anatomical regions, and
a calculation unit configured to calculate a radiation dose index value for the radiography of the region extracted by the extraction unit, based on a pixel value in the extracted region.

2. The image processing apparatus according to claim 1, wherein
the division unit adds different label numbers to the plurality of anatomical regions, and thereby divides a radiological image into a plurality of anatomical regions.

3. The image processing apparatus according to claim 1, wherein
the division unit divides the radiological image into a plurality of anatomical regions using a parameter generated through machine learning in advance.

4. The image processing apparatus according to claim 3, further comprising:
a storage unit configured to store a parameter generated through machine learning.

5. The image processing apparatus according to claim 4, wherein
the storage unit stores the parameter generated through machine learning, in association with a site.

6. The image processing apparatus according to claim 3, wherein
the extraction unit extracts at least one region from the plurality of anatomical regions, using preset information regarding a correspondence between a plurality of sites and label numbers corresponding to the plurality of sites, in accordance with an operator's instruction.

7. The image processing apparatus according to claim 6, further comprising:
a machine learning unit configured to update the parameter by performing machine learning based on predetermined image data and correct-answer division/allocation data corresponding to the predetermined image data, the predetermined image data and the correct-answer division/allocation data being set in advance,
wherein the division unit divides the radiological image into a plurality of anatomical regions using the updated parameter.

8. The image processing apparatus according to claim 7, further comprising:
a setting unit configured to update and set the correspondence information according to a plurality of anatomical regions obtained as a result of the division unit dividing a radiological image using the updated parameter.

9. The image processing apparatus according to claim 8, further comprising:
an update unit configured to update a radiation dose target value that is a target value of the radiation dose index value, using radiation dose index values calculated by the calculation unit before and after the parameter or the correspondence information is updated.

10. The image processing apparatus according to claim 3, wherein
the machine learning is machine learning that uses one of a CNN (Convolutional Neural Network), FCN (Fully Convolutional Networks), SegNet, and U-net.

11. The image processing apparatus according to claim 1, wherein
the calculation unit calculates, as a representative value, a value indicating a central tendency of the region in the radiological image extracted by the extraction unit, and calculates the radiation dose index value using the representative value.

12. The image processing apparatus according to claim 11, wherein
when a plurality of regions are extracted from the plurality of anatomical regions by the extraction unit,
the calculation unit calculates representative values for the respective regions extracted by the extraction unit, and calculates a plurality of radiation dose index values for the radiography of the plurality of extracted regions, based on the plurality of representative values.

13. An image processing method comprising:
dividing a radiological image obtained by performing radiography on a subject, into a plurality of anatomical regions:
extracting at least one region from the plurality of anatomical regions; and
calculating a radiation dose index value for the radiography of the extracted region, based on a pixel value in the extracted region.

14. A non-transitory computer-readable storage medium storing a computer program for causing a computer to execute an image processing method, the method comprising:
dividing a radiological image obtained by performing radiography on a subject, into a plurality of anatomical regions;
extracting at least one region from the plurality of anatomical regions; and
calculating a radiation dose index value for the radiography of the extracted region, based on a pixel value in the extracted region.
